(19) United States
(12) Patent Application Publication
(10) Pub. No.: US 2010/0217233 A1
(43) Pub. Date: Aug. 26, 2010

(54) METHOD AND DEVICE TO ANESTHETIZE AN AREA

(76) Inventor: Elizabeth A. Ranft, Huntersville, NC (US)

Correspondence Address: COLLEN IP, THE HOLYOKE MANHATTAN BUILDING, 80 SOUTH HIGHLAND AVENUE, OSSINING, NY, 10562, US

(21) Appl. No.: 12/390,258

(22) Filed: Feb. 20, 2009

(51) Int. Cl. A61M 19/00; A61M 5/31

(52) U.S. Cl. 604/512; 604/187

(57) ABSTRACT

Method and devices are provided to allow the application of anesthetic on an internal area of a body prior to medical procedures such as obtaining tissue for a biopsy.

Many medical procedures can cause pain to the patient while performing the procedure. Instruments are often required to be inserted into the patient's body, causing discomfort and often pain. One such procedure is obtaining an endometrial biopsy from the uterus. The manipulation of the cervix and removal of tissue from the uterus causes some level of pain for all patients. The procedure is usually done without any anesthetic being applied in the uterus.

### SUMMARY OF THE INVENTION

The present invention provides a method and devices for applying an anesthetic prior to medical procedures.

### BRIEF DESCRIPTION OF THE DRAWINGS

FIG. 1 shows a typical device for applying anesthetic.

### DESCRIPTION OF ILLUSTRATIVE EMBODIMENT

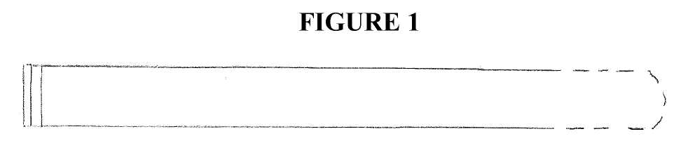

The device is intended to be fitted to the end of a syringe. The typical twist and lock means of attaching a syringe, screw mechanism or any other means can be used to connect a syringe to the device. The other end has an applicator to allow of application of the anesthetic. Various means can be used to apply the anesthetic. FIG. 1 shows a fenestrated tip as the applicator. Typically a 5-10 mL syringe filled with lidocaine 2% or lidocaine HCl 2% and epinephrine 1:100,000 would be utilized. The actual shape (straight, bend(s), etc) and dimensions of the device shown in FIG. 1 can be varied depending on the intended application, as well as the particular patient requirements.

During a typical endometrial biopsy procedure, the cervix is first stabilized by placing a tenaculum on the anterior lip of the cervix. A uterus sounder device is inserted to measure the size of the uterus. Another device such as a pipelle is then inserted and utilized to remove tissue from the uterus. The device described above can be inserted into the uterus and apply the anesthetic before the procedure for removing tissue from the uterus is performed.

In another embodiment of this invention, the sounder device itself is designed to allow the application of the anesthetic. A means is provided for a syringe to connect to the sounder device at a location on the device, which would remain outside the woman's body. The tip of the sounder device at the end intended for insertion into the uterus is provided with an applicator such as a fenestrated tip to allow wiping or spraying of the anesthetic. A hollow tunnel in the sounder device connects to syringe access point of the sounder device with its fenestrated tip. After utilizing the tenaculum to straighten the uretocervical angle, the newly designed sounder device would be inserted to into the uterus to obtain a measurement. Anesthetic is applied by pushing on the syringe while slowly pulling outward on the device thereby anesthetizing the entire uterus.

In other embodiments, the design features described above for a sounder device are applied to other medical devices. For example this method of applying anesthetic, could similarly be incorporated into the design of various gynecological instruments and uterine manipulators. The same design features may also be incorporated into devices used for procedures related to other areas of the body such as endoscopic instruments. In another embodiment, the applicator can be pre-soaked with the anesthetic or designed to allow the injected anesthetic to pass through it as device is wiped on the intended surface area.

The description above and accompanying drawings are presented as examples of the present invention. Other applications and variations of the present invention are possible to meet particular applications. For example a syringe can be replaced by other means to transfer the anesthetic onto the applicator. Thus it is recognized that departures from the disclosed embodiments may be made within the scope of the invention and that obvious modifications will occur to a person skilled in the art.

I claim:

1. An anesthetic application device comprising: a hollow tube designed for connection to a syringe at its one end; and an applicator at its other end.

2. An anesthetic application device according to claim 1 in which the applicator is a fenestrated tip.

3. An anesthetic application device comprising: a medical device intended for insertion into a body; a means to connect a syringe to the device; an applicator on the device at the end intended for insertion into the body; and a hollow tunnel within the device between a syringe connection point and the end intended for insertion into the body.

4. An anesthetic application device according to claim 3 in which the medical device intended for insertion into a body is a uterine sound device.

5. An anesthetic application device according to claim 3 in which the medical device intended for insertion into a body is a uterine manipulator device.

6. An anesthetic application device according to claim 3 in which the medical device intended for insertion into a body is an endoscopic device.

7. A method for applying anesthetic on internal parts of the body comprising: creating a device with a syringe and an applicator end, to allow application of an anesthetic; inserting the device into the area of the body to be anesthetized; using a device to transfer the anesthetic on the intended area; removing the device.
